SEQUENCE LISTING (16 sequences)

SEQ ID NO: 1 - protein, 5 aa, Synthetic construct
CREKA

SEQ ID NO: 2 - protein, 5 aa, Synthetic construct
SREKA

SEQ ID NO: 3 - protein, 5 aa, Synthetic construct
CKEKA

SEQ ID NO: 4 - protein, 5 aa, Synthetic construct
CRDKA

SEQ ID NO: 5 - protein, 5 aa, Synthetic construct
CRERA

SEQ ID NO: 6 - protein, 5 aa, Synthetic construct
CREKV

SEQ ID NO: 7 - protein, 5 aa, Synthetic construct
SKEKA

SEQ ID NO: 8 - protein, 5 aa, Synthetic construct
SRDKA

SEQ ID NO: 9 - protein, 5 aa, Synthetic construct
SRERA

SEQ ID NO: 10 - protein, 5 aa, Synthetic construct
SREKV

SEQ ID NO: 11 - protein, 5 aa, Synthetic construct
CKDKA

SEQ ID NO: 12 - protein, 5 aa, Synthetic construct
CKERA

SEQ ID NO: 13 - protein, 5 aa, Synthetic construct
CKEKV

SEQ ID NO: 14 - protein, 5 aa, Synthetic construct
CRDRA

SEQ ID NO: 15 - protein, 5 aa, Synthetic construct
CRDKV

SEQ ID NO: 16 - protein, 5 aa, Synthetic construct
CRERV

(19) United States
(12) Patent Application Publication
(10) Pub. No.: US 2025/0129184 A1
(43) Pub. Date: Apr. 24, 2025

(54) TARGETED ANTICOAGULANT

(71) Applicant: Northwestern University, Evanston, IL (US)

(72) Inventors: Guillermo A. AMEER, Chicago, IL (US); Yunxiao Zhu, Evanston, IL (US)

(21) Appl. No.: 19/000,075

(22) Filed: Dec. 23, 2024

Related U.S. Application Data
(63) Continuation of application No. 17/548,204, filed on Dec. 10, 2021, now abandoned.
Application No. 17/548,204 is a continuation of application No. 16/349,188, filed on May 10, 2019, now abandoned.
(60) Provisional application No. 62/420,930, filed on Nov. 11, 2016.

(51) Int. Cl. C07K 19/00; A61F 2/07; A61K 9/08; A61K 38/00; A61K 47/61; A61K 47/62; A61P 7/02; C07K 7/06; C07K 14/745; C08B 37/00; G01N 33/86

(52) U.S. Cl. CPC C07K 19/00; A61K 9/08; A61K 47/61; A61K 47/62; A61P 7/02; C07K 7/06; C07K 14/745; C08B 37/0081; G01N 33/86; A61F 2/07; A61K 38/00

(57) ABSTRACT

Provided herein is technology relating to anticoagulant therapies and particularly, but not exclusively, to anticoagulant compositions for localized and targeted administration and related methods and kits for treatment of a subject with a localized and targeted anticoagulant therapy.

### CROSS-REFERENCE TO RELATED APPLICATIONS

The present application is a continuation of U.S. patent application Ser. No. 17/548,204, filed Dec. 10, 2021, which is a continuation of U.S. patent application Ser. No. 16/349,188, filed May 10, 2019, which is a § 371 U.S. National Entry Application of PCT/US2017/061039, filed Nov. 10, 2017, which claims the priority benefit of U.S. Provisional Patent Application 62/420,930, filed Nov. 11, 2016, each of which is incorporated by reference in its entirety.

### SEQUENCE LISTING

The text of the computer readable sequence listing filed herewith, titled “NWEST_35092_304_SequenceListing.xml,” created Dec. 23, 2024, having a file size of 14,760 bytes, is hereby incorporated by reference in its entirety.

### FIELD

Provided herein is technology relating to anticoagulant therapies and particularly, but not exclusively, to anticoagulant compositions for localized and targeted administration and related methods and kits for treatment of a subject with a localized and targeted anticoagulant therapy.

### BACKGROUND

Patients with cardiovascular disease almost invariably receive an anticoagulant (e.g., heparin) before and after bypass surgery. The regular repeat dosing of high concentration heparin places such patients at risk of developing complications such as bleeding disorders, heparin-associated antibodies, and thromboembolic complications.

### SUMMARY

Accordingly, provided herein is technology related to a fibrin-binding-peptide modified heparin for localized targeting of an anticoagulant at the site of treatment to reduce or eliminate systemic administration of an anticoagulant and the associated higher systemic doses. In some embodiments, the fibrin-binding-peptide modified heparin is targeted to a graft site to prevent coagulation in or on the graft or device.

The technology provided herein provides several improvements and advantages relative to extant anticoagulant technologies. In particular, embodiments of the technology provided herein are compatible with current vascular grafts and medical devices, which reduce the expense of treatment by eliminating the need to pay for more expensive grafts manufactured with heparin bonded on their surfaces. Embodiments of the technology reduce the amount of anticoagulant administered to a patient, thus reducing bleeding risks for the subject. Embodiments of the technology provide for localizing heparin to a vascular graft, stent, or other medical device, which enhances endothelial cell adhesion and proliferation in the target area and, in some embodiments, improves biocompatibility. Finally, embodiments of the technology are administered in the same ways as extant anticoagulants.

The technology finds use, e.g., in any application where heparin, low molecular weight heparin, or any other anticoagulant is used to prevent clotting in medical devices such as prosthetic vascular grafts, catheters, hemodialyzers, etc. Accordingly, embodiments provide a technology for use with any medical device that contacts blood from a patient. For example, the technology finds use both for medical devices present in or on patients and finds use also for medical devices that contact patient blood outside the patient body (e.g., a device connected to the subject blood supply, e.g., dialysis machines, extracorporeal oxygenation machines, heart-lung machines, ventricular assist device, blood heaters, blood coolers, etc.). Embodiments of the technology find use with implanted blood access devices (e.g., indwelling catheter), nonimplanted blood access devices, and accessories for both implanted and nonimplanted blood access devices.

In some embodiments, the technology finds use in treating patients with synthetic heart valves, venous thrombus filters, etc. In some embodiments, the technology finds use with catheters, e.g., catheters implanted in a patient and used to access the patient's vasculature (e.g., for dialysis, administering a composition to the patient intravenously, withdrawing a patient blood sample, etc.).

Accordingly, provided herein is technology related to a composition for reducing coagulation at a target site in a subject, the composition comprising an anticoagulant and fibrin-binding peptide covalently linked to the anticoagulant. In some embodiments, the anticoagulant is heparin. In some embodiments, the fibrin-binding peptide comprises the sequence CREKA (SEQ ID NO: 1). In some embodiments, the fibrin-binding peptide is a conservative variant of CREKA (SEQ ID NO: 1). In some embodiments, the fibrin-binding peptide is SREKA (SEQ ID NO: 2); CKEKA (SEQ ID NO: 3); CRDKA (SEQ ID NO: 4); CRERA (SEQ ID NO: 5); CREKV (SEQ ID NO: 6); SKEKA (SEQ ID NO: 7); SRDKA (SEQ ID NO: 8); SRERA (SEQ ID NO: 9); SREKV (SEQ ID NO: 10); CKDKA (SEQ ID NO: 11); CKERA (SEQ ID NO: 12); CKEKV (SEQ ID NO: 13); CRDRA (SEQ ID NO: 14); CRDKV (SEQ ID NO: 15); or CRERV (SEQ ID NO: 16).

Some embodiments provide a technology related to a composition for reducing coagulation at a site in or on a medical device contacting patient blood (e.g., a medical device in the subject, a medical device on the subject, a medical device outside the subject), the composition comprising an anticoagulant and fibrin-binding peptide covalently linked to the anticoagulant. In some embodiments, the anticoagulant is heparin. In some embodiments, the fibrin-binding peptide comprises the sequence CREKA (SEQ ID NO: 1). In some embodiments, the fibrin-binding peptide is a conservative variant of CREKA (SEQ ID NO: 1). In some embodiments, the fibrin-binding peptide is SREKA (SEQ ID NO: 2); CKEKA (SEQ ID NO: 3); CRDKA (SEQ ID NO: 4); CRERA (SEQ ID NO: 5); CREKV (SEQ ID NO: 6); SKEKA (SEQ ID NO: 7); SRDKA (SEQ ID NO: 8); SRERA (SEQ ID NO: 9); SREKV (SEQ ID NO: 10); CKDKA (SEQ ID NO: 11); CKERA (SEQ ID NO: 12); CKEKV (SEQ ID NO: 13); CRDRA (SEQ ID NO: 14); CRDKV (SEQ ID NO: 15); or CRERV (SEQ ID NO: 16).

In some embodiments, the fibrin-binding peptide is covalently linked to the anticoagulant by a linker. In some embodiments, the fibrin-binding peptide and the anticoagulant are directly bonded to each other. Alternatively, in some embodiments the fibrin-binding peptide and the anticoagulant are joined using a linker. The linker can be peptidic or non-peptidic in nature. In some embodiments, the linker is an all-carbon chain, or, in some embodiments, the linker comprises heteroatoms such as, e.g., oxygen, nitrogen, sulfur, and phosphorus. In some embodiments, the linker comprises a PEG (polyether) region. The linker can be a linear or branched chain, or can include structural elements such as phenyl ring(s), non-aromatic carbocyclic or heterocyclic ring(s), double or triple bond(s), and the like. In some embodiments, a linker is substituted with halo, pseudohalo, alkyl, alkenyl, alkynyl, alkoxy, cycloalkyl, aryl, amino, hydroxy, carbonyl, alkoxycarbonyl, and hydroxycarbonyl groups. In some embodiments, the linker includes functional groups (e.g., multiple functional groups) that are appropriate for conjugation to one or more fibrin-binding and anticoagulants. In some embodiments, the linker is, e.g., N-B-maleimidopropionic acid hydrazide.

In some embodiments, the linker comprises multiple functional groups for attachment of one or more fibrin-binding and anticoagulants.

In some embodiments, the composition further comprises a physiologically appropriate solution for administration to a subject. In some embodiments, the composition further comprises a fibrin bound to the fibrin-binding peptide. In some embodiments, the composition further comprises a medical device. In some embodiments, the subject has cardiovascular disease. In some embodiments, the subject has had or requires bypass surgery. In some embodiments, the target site is at or near a location of a medical device in the subject. In some embodiments, the target site is at or near a location of a prosthetic vascular graft, catheter, or hemodialyzer in the subject.

Further embodiments relate to a method for treating a subject in need of anticoagulation therapy, the method comprising administering to the subject a composition comprising a fibrin-binding peptide covalently linked to an anticoagulant. In some embodiments, the anticoagulant is heparin. In some embodiments, the fibrin-binding peptide comprises the sequence CREKA (SEQ ID NO: 1). In some embodiments, the composition comprises heparin-CREKA. In some embodiments, the composition is administered at a dose that is lower than a dose of heparin appropriate for administration to the subject for the same therapy. In some embodiments, the subject has a cardiovascular disease. In some embodiments, the methods further comprise identifying the subject as requiring bypass surgery. In some embodiments, the target site is at or near a location of a medical device in the subject. In some embodiments, the target site is at or near a location of a prosthetic vascular graft, catheter, or hemodialyzer in the subject.

Some embodiments relate to applying a composition as described herein (e.g., a composition comprising a fibrin-binding peptide covalently linked to an anticoagulant) to a medical device (e.g., a medical device that contacts or may contact patient blood (e.g., a medical device in the subject, a medical device on the subject, a medical device outside the subject)). Some embodiments relate to contacting a medical device (e.g., a medical device that contacts or may contact patient blood (e.g., a medical device in the subject, a medical device on the subject, a medical device outside the subject)) with a composition as described herein (e.g., a composition comprising a fibrin-binding peptide covalently linked to an anticoagulant).

Still further embodiments provide a method for producing a fibrin-binding peptide covalently linked to an anticoagulant, the method comprising activating a carboxyl group on an anticoagulant; covalently linking a linker to the activated carboxyl group; and covalently linking a fibrin-binding peptide to the linker. In some embodiments, the anticoagulant is heparin. In some embodiments, the linker is a bifunctional linker comprising an amide and a maleimide. In some embodiments, the fibrin-binding peptide is CREKA (SEQ ID NO: 1). In some embodiments, activating the carboxyl group on the anticoagulant comprises use of a carbodiimide reaction. In some embodiments, activating the carboxyl group on the anticoagulant comprises use of 1-ethyl-3-(-3-dimethylaminopropyl) carbodiimide hydrochloride. In some embodiments, the bifunctional linker is N-B-maleimidopropionic acid hydrazide. In some embodiments, covalently linking the fibrin-binding peptide to the linker comprises use of a thiol-maleimide click reaction. In some embodiments, the fibrin-binding peptide is a conservative variant of CREKA (SEQ ID NO: 1). In some embodiments, the fibrin-binding peptide is SREKA (SEQ ID NO: 2); CKEKA (SEQ ID NO: 3); CRDKA (SEQ ID NO: 4); CRERA (SEQ ID NO:

5); CREKV (SEQ ID NO: 6); SKEKA (SEQ ID NO: 7); SRDKA (SEQ ID NO: 8); SRERA (SEQ ID NO: 9); SREKV (SEQ ID NO: 10); CKDKA (SEQ ID NO: 11); CKERA (SEQ ID NO: 12); CKEKV (SEQ ID NO: 13); CRDRA (SEQ ID NO: 14); CRDKV (SEQ ID NO: 15); or CRERV (SEQ ID NO: 16).

In additional embodiments, the technology provides a reaction mixture comprising an anticoagulant and a linker. In some embodiments, the anticoagulant comprises an activated group. In some embodiments, the anticoagulant comprises an activated carboxyl. In some embodiments, the linker is covalently attached to the anticoagulant. In some embodiments, reaction mixture further comprises a fibrin-binding peptide. In some embodiments, the reaction mixture comprises an anticoagulant linked to a fibrin-binding peptide by a linker. In some embodiments, the anticoagulant is heparin. In some embodiments, the fibrin-binding peptide comprises the sequence CREKA (SEQ ID NO: 1). In some embodiments, the fibrin-binding peptide is a conservative variant of CREKA (SEQ ID NO: 1). In some embodiments, the fibrin-binding peptide is SREKA (SEQ ID NO: 2); CKEKA (SEQ ID NO: 3); CRDKA (SEQ ID NO: 4); CRERA (SEQ ID NO: 5); CREKV (SEQ ID NO: 6); SKEKA (SEQ ID NO: 7); SRDKA

(SEQ ID NO: 8); SRERA (SEQ ID NO: 9); SREKV (SEQ ID NO: 10); CKDKA (SEQ ID NO: 11); CKERA (SEQ ID NO: 12); CKEKV (SEQ ID NO: 13); CRDRA (SEQ ID NO: 14); CRDKV (SEQ ID NO: 15); or CRERV (SEQ ID NO: 16). In some embodiments, the reaction mixture comprises1-ethyl-3-(-3-dimethyl-aminopropyl) carbodiimide hydrochloride. In some embodiments, the linker is N-β-maleimidopropionic acid hydrazide.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a bar plot showing the synthetic efficiency for three ratios of 1-ethyl-3-(-3-dimethylaminopropyl) carbodiimide hydrochloride (EDC) to heparin (5 to 1, 10 to 1, and 20 to 1) used in a carbodiimide reaction to activate carboxyl groups on heparin.

FIG. 2 is a bar plot showing the synthetic efficiency for three ratios of bifunctional linker (N-β-maleimidopropionic acid hydrazide, BMPH) to activated heparin (5 to 1, 10 to 1, and 20 to 1) used to provide maleimide groups for attachment to the fibrin-binding peptide.

FIG. 3 is a bar plot showing the synthetic efficiency of maleimide conjugation of a linker maleimide group to a fibrin-binding peptide cysteine sulfhydryl.

FIG. 4 is a bar plot showing the anti-coagulant effect of heparin-CREKA relative to conventional heparin as a function of time. Higher absorbance represents more unclotted red blood cells and better anti-coagulation effect.

FIG. 5 is a bar plot showing the anticoagulation activity of the heparin-CREKA compounds relative to conventional heparin as a function of concentration. Heparin-CREKA treatment significantly reduced the amount of adhered platelets at a lower concentration than the addition of conventional heparin.

FIG. 6 is a pair of bar plots showing the binding specificity of CREKA peptides towards fibrin relative to the binding of a scrambled CERAK peptide.

FIG. 7, Panels A-D, is a series of photographs showing the exposure of rat aorta (Panel A), excision of an aorta segment (Panel B), and placement of an ePTFE graft (Panel C). Panel D is a diagram showing the segments of the graft that were tested after removal of the graft from the host.

FIG. 8, Panels A-C, is a series of images from analysis of the segmented grafts.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to anticoagulant therapies and particularly, but not exclusively, to anticoagulant compositions for localized and targeted administration and related methods and kits for treatment of a subject with a localized and targeted anticoagulant therapy.

Beneficial therapeutic effects of pharmaceuticals are derived from pharmaceutical activity at the site of drug action (Pharmacology-Drug Actions and Reactions, Ruth R. Levine, Parthenon Publishing, 6^{th} Edition). For non-targeted anticoagulant therapies, high systemic concentration of the anticoagulant is required to maintain a certain plasma anticoagulant concentration for a non-targeted anticoagulant to reach the site of action by passive diffusion and reside there for a long enough period of time to be therapeutically beneficial. The compositions described herein provide compounds that are localized and retained at the site of action without relying solely on passive diffusion to deliver the therapeutic agent to the site of action. These compounds thus agents effectively deliver anticoagulants to the target sites, thus raising the local anticoagulant concentration at the desired site of drug action. Furthermore, the compounds described herein are retained in the tissues at the target site for a longer period of time than non-targeted anticoagulants due to their binding to fibrin within or around the target site and target tissues, thus allowing for longer durations of drug action. This “concentrating” or “homing” effect reduces the need to maintain higher plasma levels of the anticoagulants due to metabolism of the active drug, e.g., by circulating degradation enzymes, and in tissues through which the blood supply circulates, e.g., the liver, thereby improving safety profiles relative to non-targeted therapeutics. In some embodiments, the technology provided herein finds use in “rescuing” anticoagulant that fail clinical tests by reducing unacceptable systemic toxicity, such as systemic bleeding of some currently disfavored non-targeted or systemic therapeutic agents, to within acceptable limits.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

A “fibrin-binding peptide” or “fibrin-targeting peptide” refers to a peptide or portion of a protein or polypeptide that specifically binds to fibrin or to a soluble or insoluble fragment of fibrin. A fibrin fragment may have a structure or characteristic exhibited by fibrin. In some embodiments, the term fibrin-binding peptide or fibrin-targeting peptide as used herein does not encompass antibodies and antibody fragments such as, e.g., recombinant, chimeric, humanized, monoclonal, or polyclonal antibodies or antibody fragments, including single chains that are specific for fibrin or a soluble or insoluble fragment of fibrin.

The term “reducing coagulation” or “inhibiting coagulation” or “minimizing coagulation” refers to preventing or reducing the rate of coagulation, growth of a clot, or thrombus accretion in any dimension. The treatment of coagulation may also include reducing the size of a clot or thrombus in any dimension. The size of the clot or thrombus may be reduced by an amount of 5% or more (e.g., 10%, 20%, 30%, 40%, 45%, 50%, 55%, 60%, 65%, 70%, 75%, 80%, 85%, 90%, 92%, 95% or more). The patency of a blood vessel and/or medical device (e.g., graft) may be maintained or increased, e.g., as assessed by angiography, including X-ray angiography, MR angiography, ultrasound, or doppler flow. By maintaining or prolonging vessel and/or medical device patency, a longer time window is provided in which intervention may be efficacious. The treatment of coagulation may also be prophylactic, used for example in combination with percutaneous coronary or peripheral artery intervention, or elective surgery, e.g. athroplasty.

The term “peptidomimetic,” as used herein, means a peptide-like molecule that has the activity of the peptide upon which it is structurally based. Such peptidomimetics include chemically modified peptides, peptide-like molecules containing non-naturally occurring amino acids, and peptoids; peptidomimetic have the same activity as the peptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff, John Wiley & Sons 1995), pages 803-861).

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.), and the like.

As used herein, the term “co-administration” refers to the administration of at least two agents or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent.

As used herein, the term “pharmaceutical composition” refers to the combination of an active agent with a carrier, inert or active, making the composition especially suitable for therapeutic use. As used herein, the term “pharmaceutical composition” refers to the combination of an active agent with, as desired, a carrier, inert or active, making the composition especially suitable for diagnostic or therapeutic use in vitro, in vivo, or ex vivo.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (e.g., minimize or lessen) the targeted pathologic condition or disorder. Those in need of treatment include those already with the disorder as well as those prone to have the disorder or those in whom the disorder is to be prevented.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present technology.

The terms “protein” and “polypeptide” refer to compounds comprising amino acids joined via peptide bonds and are used interchangeably. A “protein” or “polypeptide” encoded by a gene is not limited to the amino acid sequence encoded by the gene, but includes post-translational modifications of the protein.

Where the term “amino acid sequence” is recited herein to refer to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “portion” when used in reference to a protein (as in “a portion of a given protein”) refers to fragments of that protein. The fragments may range in size from four amino acid residues to the entire amino sequence minus one amino acid (for example, the range in size includes 4, 5, 6, 7, 8, 9, 10, or 11 . . . amino acids up to the entire amino acid sequence minus one amino acid).

“Native” proteins or polypeptides refer to proteins or polypeptides recovered from a source occurring in nature.

The terms “analog”, “fragment”, “derivative”, and “variant”, when referring to the peptides, compounds, and synthetic constructs of this invention means analogs, fragments, derivatives, and variants of the peptides, compounds, and synthetic constructs that retain substantially the same biological function or activity, as described further below.

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (i.e., additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “domain” when used in reference to a polypeptide refers to a subsection of the polypeptide which possesses a unique structural and/or functional characteristic; typically, this characteristic is similar across diverse polypeptides. The subsection typically comprises contiguous amino acids, although it may also comprise amino acids which act in concert or which are in close proximity due to folding or other configurations. Examples of a protein domain include the transmembrane domains, and the glycosylation sites.

As used herein, “modulation” or “to modulate” means either an increase (stimulation) or a decrease (inhibition) in the expression and/or activity of a gene and/or a gene product. For example, expression may be inhibited to potentially prevent tumor proliferation. “Modulation” may also be spatial or temporal modulation, e.g., a change in the time or location where expression or activity occurs.

The term “composition comprising” a given compound and/or polypeptide refers broadly to any composition containing the given compound and/or polypeptide.

A peptide that “specifically binds to” or is “specific for” a target is a peptide that binds to that target without substantially binding to other non-target molecules, suramolecular structures, etc. Accordingly, the term “specific binding affinity” as used herein, refers to the capacity of a molecule to bind a particular biological component (a “target”) to a greater degree than other components (a “non-target”). The specific binding affinity of a molecule for a target can be expressed in terms of the equilibrium dissociation constant “Kd.”

As used herein, the term “target site” refers to a site within a subject or a tissue (in vivo or in vitro) where it is desired to provide or deliver an anticoagulant (e.g., increase the concentration and/or amount of anticoagulant at the target site relative to the concentration and/or amount of anticoagulant in the absence of treatment), e.g., a site at which coagulation is present, a site in which coagulation is suspected to occur in the future, a site in which coagulation would be detrimental to the subject, a site at which coagulation is predicted to occur based on knowledge in the art concerning medical interventions performed at the site, etc. The term “locus” refers to any portion of the tissue or cells itself in proximity to the target site (e.g., surrounding the site), including normal and/or diseased portions. In some embodiments, the locus comprises a medical device (e.g., a graft). In some embodiments, the site comprises a medical device (e.g., a graft). As such, the methods of the present invention are used in some embodiments to target an agent toward a target site in a tissue. A target site may be the entire tissue, or may be a portion of the tissue, such as blood vessels, or may be individual or groups of cells, in vivo or in vitro, or in situ, that make up the tissue. The phrases targeted site and targeted tissue are used interchangeably herein.

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, “inhibitor” refers to a molecule which eliminates, minimizes, or decreases activity, e.g., the biological, enzymatic, chemical, or immunological activity, of a biological molecule.

As used herein the term “disease” refers to a deviation from the condition regarded as normal or average for members of a species, and which is detrimental to an affected individual under conditions that are not inimical to the majority of individuals of that species (e.g., diarrhea, nausea, fever, pain, inflammation, etc.).

As used herein, “therapeutically effective dose” refers to an amount of a therapeutic agent sufficient to bring about a beneficial or desired clinical effect. Said dose can be administered in one or more administrations. However, the precise determination of what would be considered an effective dose may be based on factors individual to each patient, including, but not limited to, the patient's age, size, type or extent of disease, stage of the disease, route of administration, the type or extent of supplemental therapy used, ongoing disease process, and type of treatment desired (e.g., aggressive vs. conventional treatment).

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the terms “pharmaceutically acceptable” or “pharmacologically acceptable” refer to compositions that do not substantially produce adverse reactions, e.g., toxic, allergic, or immunological reactions, when administered to a subject.

As used herein, “carriers” include pharmaceutically acceptable carriers, excipients, or stabilizers which are nontoxic to the cell or mammal being exposed thereto at the dosages and concentrations employed. Often the physiologically acceptable carrier is an aqueous pH-buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants.

As used herein, the terms “patient” or “subject” refer to organisms to be treated by the compositions of the present technology or to be subject to various tests provided by the technology. The term “subject” includes animals, preferably mammals, including humans. In a preferred embodiment, the subject is a primate. In an even more preferred embodiment, the subject is a human.

As used herein, “activating” a chemical group refers to increasing its reactivity for a particular chemical reaction such that the reactivity is high enough to be useful, e.g., high enough to provide an adequate yield with minimal or no by-products.

As used herein, “click chemistry” refers to a chemical reaction or series of reactions that produce a desirable chemical yield, is physiologically stable, and exhibits a large thermodynamic driving force that favors a “spring-loaded” reaction that yields a single product. See, e.g., Kolb, Finn, Sharpless (2001) “Click Chemistry: Diverse Chemical Function from a Few Good Reactions”, Angewandte Chemie International Edition 40(11): 2004-2021, incorporated herein by reference in its entirety.

### Description

Some embodiments of the technology provide a targeted and localized anticoagulation therapy. In particular, embodiments of the technology provide a short fibrin-binding peptide (CREKA, SEQ ID NO: 1) that is covalently attached to a heparin molecule.

Embodiments of the technology provide an anticoagulant linked to a fibrin-binding peptide. The technology is not limited in the anticoagulant used; the technology is not limited in the fibrin-binding peptide used. For example, in some embodiments the anticoagulant is heparin or a modification or derivative thereof; in some embodiments, the fibrin-binding peptide is a CREKA (SEQ ID NO: 1) peptide or a modification or derivative thereof.

In some embodiments, the anticoagulant is any anticoagulant known to those of ordinary skill in the art, e.g., coumarins and indandiones; factor Xa inhibitors; heparins; and thrombin inhibitors. In some embodiments, the anticoagulant is warfarin (e.g., COUMADIN or MAREVAN); dabigatran (e.g., PRADAXA); apixaban (e.g., ELIQUIS); or rivaroxaban (e.g., XARELTO). In some embodiments, the technology comprises use of an antiplatelet drugs and/or thrombolytic drugs that are linked to the fibrin-binding peptide.

Anticoagulants affect, directly or indirectly, enzymes, cofactors, or polypeptides involved in the thrombin coagulation cascade. For example, in some embodiments, an inhibits enzymes and/or polypeptides that activate thrombin, or can inhibit thrombin directly. Examples include thrombin inhibitor (e.g., melagatran), Factor Xa inhibitor, tissue factor inhibitor, Factor VIIa inhibitor, Factor IXa inhibitor, Factor Va inhibitor, Factor XIa inhibitor, Factor XIIa inhibitor, TAFIα inhibitor, α2-antiplasmin inhibitor, PAI-1 inhibitor, PAI-2 inhibitor, PAI-3 inhibitor, prothrombinase inhibitor, tick anticoagulation peptide, protein C, warfarin, heparin, lepirudin, aspirin, ticlopidine, clopidogrel, tirofiban, and eptifibatide.

The CREKA (SEQ ID NO: 1) peptide is a peptide comprising the amino acids cysteine, arginine, glutamic acid, lysine, and alanine. In some embodiments, the CREKA (SEQ ID NO: 1) peptide is a peptide comprising an ordered sequence of amino acids that is ordered as cysteine, arginine, glutamic acid, lysine, and alanine from the N-terminal end to the C-terminal end.

In some embodiments, the technology provides an isolated peptide comprising the amino acid sequence CREKA (SEQ ID NO: 1), or a conservative variant or peptidomimetic thereof. As used herein in reference to a specified amino acid sequence, a “conservative variant” is a sequence in which a first amino acid is replaced by another amino acid or amino acid analog having at least one biochemical property similar to that of the first amino acid; similar properties include, for example, similar size, charge, hydrophobicity or hydrogen-bonding capacity.

As an example, in some embodiments a conservative variant is a sequence in which a first uncharged polar amino acid is conservatively substituted with a second (non-identical) uncharged polar amino acid such as cysteine, serine, threonine, tyrosine, glycine, glutamine, or asparagine or an analog thereof. A conservative variant also can be a sequence in which a first basic amino acid is conservatively substituted with a second basic amino acid such as arginine, lysine, histidine, 5-hydroxylysine, N-methyllysine, or an analog thereof. Similarly, in some embodiments a conservative variant is a sequence in which a first hydrophobic amino acid is conservatively substituted with a second hydrophobic amino acid such as alanine, valine, leucine, isoleucine, proline, methionine, phenylalanine, or tryptophan or an analog thereof. In the same way, in some embodiments a conservative variant is a sequence in which a first acidic amino acid is conservatively substituted with a second acidic amino acid such as aspartic acid or glutamic acid or an analog thereof; a sequence in which an aromatic amino acid such as phenylalanine is conservatively substituted with a second aromatic amino acid or amino acid analog, for example, tyrosine; or a sequence in which a first relatively small amino acid such as alanine is substituted with a second relatively small amino acid or amino acid analog such as glycine or valine or an analog thereof.

Thus, as non-limiting examples, the technology includes conservative variants of CREKA (SEQ ID NO: 1), e.g., SREKA (SEQ ID NO: 2); CKEKA (SEQ ID NO: 3); CRDKA (SEQ ID NO: 4); CRERA (SEQ ID NO: 5); CREKV (SEQ ID NO: 6); SKEKA (SEQ ID NO: 7); SRDKA (SEQ ID NO: 8); SRERA (SEQ ID NO: 9); SREKV (SEQ ID NO: 10); CKDKA (SEQ ID NO: 11); CKERA (SEQ ID NO: 12); CKEKV (SEQ ID NO: 13); CRDRA (SEQ ID NO: 14); CRDKV (SEQ ID NO: 15); and CRERV (SEQ ID NO: 16). It is understood that conservative variants of CREKA (SEQ ID NO: 1) encompass sequences comprising one, two, three, four, or more amino acid substitutions relative to SEQ ID NO: 1 and that such variants can include naturally and non-naturally occurring amino acid analogs. In some embodiments, the fibrin-binding peptide is a peptide that comprises the CREKA or conservative variant thereof and additional amino acids, e.g., at the N-terminal and/or C-terminal ends.

Some embodiments provide a peptidomimetic of CREKA. A variety of peptidomimetics are known in the art including, for example, peptide-like molecules that comprise a constrained amino acid, a non-peptide component that mimics peptide secondary structure, or an amide bond isostere. A peptidomimetic that comprises a constrained, non-naturally occurring amino acid can include, for example, an α-methylated amino acid; α,α-dialkylglycine, or α-aminocycloalkane carboxylic acid; an Nα-Cα cyclized amino acid; an Na-methylated amino acid; a β-or γ-amino cycloalkane carboxylic acid; an α,β-unsaturated amino acid; a β,β-dimethyl or β-methyl amino acid; a β-substituted-2,3-methano amino acid; an N-Cε or Cα-Cδ cyclized amino acid; a substituted proline, or another amino acid mimetic. A peptidomimetic that mimics peptide secondary structure can comprise, for example, a non-peptidic β-turn mimic; γ-turn mimic; mimic of β-sheet structure; or mimic of helical structure, each of which is well known in the art. A peptidomimetic also can be a peptide-like molecule which comprises, for example, an amide bond isostere such as a retro-inverso modification; reduced amide bond; methylenethioether or methylene-sulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. One skilled in the art understands that these and other peptidomimetics are encompassed within the meaning of the term “peptidomimetic” as used herein.

Methods for identifying a peptidomimetic are well known in the art and include, for example, the screening of databases that contain libraries of potential peptidomimetics. As an example, the Cambridge Structural Database contains a collection of greater than 300,000 compounds that have known crystal structures (Allen et al., Acta Crystalloqr. Section B, 35:2331 (1979)). This structural depository is continually updated as new crystal structures are determined and can be screened for compounds having suitable shapes, for example, the same shape as a peptide of the technology provided, as well as potential geometrical and chemical complementarity to a target molecule. Where no crystal structure of a peptide of the technology or a target molecule that binds the peptide is available, a structure can be generated using, for example, the program CONCORD (Rusinko et al., J. Chem. Inf. Comput. Sci. 29:251 (1989)). Another database, the Available Chemicals Directory (Molecular Design Limited, Information Systems; San Leandro Calif.), contains about 100,000 compounds that are commercially available and also can be searched to identify potential peptidomimetics of a peptide of the invention, for example, with activity in selectively homing to tumor vasculature and selectively binding to collagen.

In some embodiments, the fibrin binding peptide is attached to the anticoagulant by a novel method using a bifunctional linker. In particular embodiments, the bifunctional linker covalently links peptides with a terminal Cys residue to one or more carboxyl groups on the heparin molecule. In some embodiments, this method maximizes the number of peptides that are conjugated in a specific orientation, thus leaving the active sites within the peptide backbone and the side chains available for interaction with fibrin on the graft while the anticoagulant activity of the heparin is maintained.

In some embodiments, the resulting heparin-CREKA compound is water soluble. In some embodiments, the heparin-CREKA compound has an affinity for fibrin. In some embodiments, the heparin-CREKA compound prevents clotting on surfaces for extended amounts of time. The technology finds use in a clinical setting in which the heparin-CREKA localizes the anticoagulant activity at the targeted location where it is needed. In some embodiments, heparin-CREKA is used in a combination therapy, e.g., with other anticoagulants. Accordingly, the various embodiments of technology improve the efficacy of anticoagulation procedures and the function of medical devices.

Synthetic grafts, such as expanded polytetrafluoroethylene (ePTFE) grafts, are commonly used as prostheses in bypass surgeries to treat atherosclerosis and other vascular dysfunctions; however, success rates using synthetic grafts are variable. One prominent issue with synthetic grafts is that their inside surface, where blood flow occurs, does not comprise endothelial cells to prevent the formation of fibrin clots. Fibrin is the main component of blood clots, and once it deposits onto the graft surface, propagation of clotting worsens by continuous contact with blood. Blood clotting on vascular grafts is dangerous because it can cause inflammation and limit or even block blood flow, thus leading to graft failure.

Patients who receive vascular grafts are often administered heparin to prevent the formation of fibrin clots on the intimal surface of the graft. Heparin is usually administered intravenously as a short-term and fast-acting treatment, especially directly after vascular bypass surgeries. However, heparin is also known to cause internal bleeding when administered systemically, and thus heparin administration is associated with long-term many short-term and long-term risks. Recent studies have explored direct functionalization of graft surfaces with heparin; however, the chemically linked heparin cannot be replenished on the graft surfaces once it was depleted or otherwise removed and chemical treatment changes the mechanical and surface properties of grafts.

In contrast, embodiments of the technologies described herein comprising use of heparin-CREKA provide a fibrin-selective, low-dose anticoagulant alternative to high frequency, larger dosages of heparin for the prevention of clotting, e.g., in vascular grafts and medical devices that are prone to generate fibrin at their surfaces in contact with blood. Side effects such as premature clotting in the device as well as patient bleeding are avoided by targeting the heparin or other anticoagulant drug to the device or pro-thrombogenic surface.

Embodiments of the technology comprise use of any medical device. In some embodiments, the medical device is an implantable device such as a vascular graft, endoprosthesis, or stent. In some embodiments, other medical devices are used, e.g., catheters. Some embodiments comprise use of a vascular graft that comprises a hollow tubular body, e.g., having an inner and an outer surface (e.g., an inner hydrophobic surface and/or an outer hydrophobic surface). In some embodiments, the technology comprises use of a small caliber vascular graft and in some embodiments the technology comprises use of an ePTFE vascular graft. As used herein, the term “vascular graft” is meant to include endoprostheses that are generally introduced via catheter.

In some embodiments of the technology, a medical device is provided that comprises a heparin-CREKA coating over a body fluid contacting surface of the medical device for contacting body fluids.

In some embodiments of the technology, there is provided a surface-modified implantable material comprising a treated surface that is antithrombogenic when exposed to a body fluid over extended periods of time.

The technology is not limited in the medical device with which the compositions described herein are used. For example, in some embodiments, the medical device is a medical device that contacts patient blood (e.g., either a medical device inside the patient's body, a medical device that penetrates the patient's body (e.g., a medical device comprising a portion that is inside the patient's body and a portion that is outside the patient's body), and/or a medical device that is outside the patient's body). Exemplary, non-limiting, medical devices that find use with the compositions described herein include prosthetic vascular grafts, catheters, hemodialyzers, extracorporeal oxygenation machines, heart-lung machines, ventricular assist device, blood heaters, blood coolers, etc.

In some embodiments, the technology relates to administration of a compound comprising a fibrin-binding peptide linked to an anticoagulant, e.g., a heparin-CREKA compound, to a subject. In some embodiments, the technology relates to contacting a medical device with a compound comprising a fibrin-binding peptide linked to an anticoagulant, e.g., a heparin-CREKA compound. It is generally contemplated that embodiments of a compound comprising a fibrin-binding peptide linked to an anticoagulant, e.g., a heparin-CREKA compound, are formulated for administration to a mammal, and especially to a human with a condition that is responsive to the administration of such compounds. Therefore, where contemplated compounds are administered in a pharmacological composition, it is contemplated that the contemplated compounds are formulated in admixture with a pharmaceutically acceptable carrier. For example, contemplated compounds can be administered orally as pharmacologically acceptable salts or intravenously in a physiological saline solution (e.g., buffered to a pH of about 7.2 to 7.5). Conventional buffers such as phosphates, bicarbonates, or citrates can be used for this purpose. Of course, one of ordinary skill in the art may modify the formulations within the teachings of the specification to provide numerous formulations for a particular route of administration. In particular, contemplated compounds may be modified to render them more soluble in water or other vehicle, which, for example, may be easily accomplished with minor modifications (salt formulation, esterification, etc.) that are well within the ordinary skill in the art. It is also well within the ordinary skill of the art to modify the route of administration and dosage regimen of a particular compound to manage the pharmacokinetics of the present compounds for maximum beneficial effect in a patient.

In certain pharmaceutical dosage forms, prodrug forms of contemplated compounds may be formed for various purposes, including reduction of toxicity, increasing the organ or target cell specificity, etc. Among various prodrug forms, acylated (e.g., acetylated or other) derivatives, pyridine esters, and various salt forms of the present compounds are preferred. One of ordinary skill in the art will recognize how to modify the present compounds to prodrug forms to facilitate delivery of active compounds to a target site within the host organism or patient. One of ordinary skill in the art will also take advantage of favorable pharmacokinetic parameters of the prodrug forms, where applicable, in delivering the present compounds to a targeted site within the host organism or patient to maximize the intended effect of the compound. Similarly, it should be appreciated that contemplated compounds may also be metabolized to their biologically active form and all metabolites of the compounds herein are therefore specifically contemplated. In addition, contemplated compounds (and combinations thereof) may be administered in combination with yet further agents.

With respect to administration to a subject, it is contemplated that the compounds are administered in a pharmaceutically effective amount. One of ordinary skill recognizes that a pharmaceutically effective amount varies depending on the therapeutic agent used, the subject's age, condition, and sex, and on the extent of the disease in the subject. Generally, the dosage should not be so large as to cause adverse side effects, such as hyperviscosity syndromes, pulmonary edema, congestive heart failure, and the like. The dosage can also be adjusted by the individual physician or veterinarian to achieve the desired therapeutic goal.

As used herein, the actual amount encompassed by the term “pharmaceutically effective amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication, and other factors that those skilled in the art will recognize.

Pharmaceutical compositions (e.g., for administering to a subject, for contacting a medical device) preferably comprise one or more embodiments of a compound comprising a fibrin-binding peptide linked to an anticoagulant, e.g., a heparin-CREKA compound, associated with one or more pharmaceutically acceptable carriers, diluents, or excipients. Pharmaceutically acceptable carriers are known in the art such as those described in, for example, Remingtons Pharmaceutical Sciences, Mack Publishing Co. (A. R. Gennaro edit. 1985), explicitly incorporated herein by reference for all purposes.

Accordingly, in some embodiments, a compound comprising a fibrin-binding peptide linked to an anticoagulant, e.g., a heparin-CREKA compound, is formulated as a tablet, a capsule, a time release tablet, a time release capsule; a time release pellet; a slow release tablet, a slow release capsule; a slow release pellet; a fast release tablet, a fast release capsule; a fast release pellet; a sublingual tablet; a gel capsule; a microencapsulation; a transdermal delivery formulation; a transdermal gel; a transdermal patch; a sterile solution; a sterile solution prepared for use as an intramuscular or subcutaneous injection, for use as a direct injection into a targeted site, or for intravenous administration; a solution prepared for rectal administration; a solution prepared for administration through a gastric feeding tube or duodenal feeding tube; a suppository for rectal administration; a liquid for oral consumption prepared as a solution or an elixir; a topical cream; a gel; a lotion; a tincture; a syrup; an emulsion; or a suspension.

In some embodiments, the time release formulation is a sustained-release, sustained-action, extended-release, controlled-release, modified release, or continuous-release mechanism, e.g., the composition is formulated to dissolve quickly, slowly, or at any appropriate rate of release of the compound comprising a fibrin-binding peptide linked to an anticoagulant, e.g., a heparin-CREKA compound, over time.

In some embodiments, the compositions are formulated so that the active ingredient is embedded in a matrix of an insoluble substance (e.g., various acrylics, chitin) such that the dissolving drug finds its way out through the holes in the matrix, e.g., by diffusion. In some embodiments, the formulation is enclosed in a polymer-based tablet with a laser-drilled hole on one side and a porous membrane on the other side. Stomach acids push through the porous membrane, thereby pushing the drug out through the laser-drilled hole. In time, the entire drug dose releases into the system while the polymer container remains intact, to be excreted later through normal digestion. In some sustained-release formulations, the compound comprising a fibrin-binding peptide linked to an anticoagulant, e.g., a heparin-CREKA compound, dissolves into the matrix and the matrix physically swells to form a gel, allowing the drug to exit through the gel's outer surface. In some embodiments, the formulations are in a micro-encapsulated form, e.g., which is used in some embodiments to produce a complex dissolution profile. For example, by coating the compound comprising a fibrin-binding peptide linked to an anticoagulant, e.g., a heparin-CREKA compound, around an inert core and layering it with insoluble substances to form a microsphere, some embodiments provide more consistent and replicable dissolution rates in a convenient format that is combined in particular embodiments with other controlled (e.g., instant) release pharmaceutical ingredients, e.g., to provide a multipart gel capsule.

In some embodiments, the pharmaceutical preparations and/or formulations of the technology are provided in particles. “Particles” as used herein means nano-or microparticles (or in some instances larger) that consist of, in whole or in part, a compound comprising a fibrin-binding peptide linked to an anticoagulant, e.g., a heparin-CREKA compound described herein. The particles may contain the preparations and/or formulations in a core surrounded by a coating, including, but not limited to, an enteric coating. The preparations and/or formulations also may be dispersed throughout the particles. The preparations and/or formulations also may be adsorbed into the particles. The particles may be of any order release kinetics, including zero order release, first order release, second order release, delayed release, sustained release, immediate release, and any combination thereof, etc. The particle may include, in addition to the preparations and/or formulations, any of those materials routinely used in the art of pharmacy and medicine, including, but not limited to, erodible, nonerodible, biodegradable, or nonbiodegradable material or combinations thereof. The particles may be microcapsules which contain the formulation in a solution or in a semi-solid state. The particles may be of any shape.

Both non-biodegradable and biodegradable polymeric materials can be used in the manufacture of particles for delivering the preparations and/or formulations. Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein by reference. These include polyhyaluronic acids, casein, gelatin, glutin, polyanhydrides, polyacrylic acid, alginate, chitosan, poly(methyl methacrylate), poly(ethyl methacrylate), poly(butyl methacrylate), poly (isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The technology also provides methods for preparing stable pharmaceutical preparations containing aqueous solutions of a compound comprising a fibrin-binding peptide linked to an anticoagulant, e.g., a heparin-CREKA compound, to inhibit formation of degradation products. A solution is provided that contains a compound comprising a fibrin-binding peptide linked to an anticoagulant, e.g., a heparin-CREKA compound, and at least one degradation inhibiting agent. The solution is processed under at least one sterilization technique prior to and/or after terminal filling the solution in the sealable container to form a stable pharmaceutical preparation. The present formulations may be prepared by various methods known in the art so long as the formulation is substantially homogenous, e.g., the pharmaceutical is distributed substantially uniformly within the formulation. Such uniform distribution facilitates control over drug release from the formulation.

In some embodiments, a compound comprising a fibrin-binding peptide linked to an anticoagulant, e.g., a heparin-CREKA compound, is formulated with a buffering agent. The buffering agent may be any pharmaceutically acceptable buffering agent. Buffer systems include citrate buffers, acetate buffers, borate buffers, and phosphate buffers. Examples of buffers include citric acid, sodium citrate, sodium acetate, acetic acid, sodium phosphate and phosphoric acid, sodium ascorbate, tartartic acid, maleic acid, glycine, sodium lactate, lactic acid, ascorbic acid, imidazole, sodium bicarbonate and carbonic acid, sodium succinate and succinic acid, histidine, and sodium benzoate and benzoic acid.

In some embodiments, a compound comprising a fibrin-binding peptide linked to an anticoagulant, e.g., a heparin-CREKA compound, is formulated with a chelating agent. The chelating agent may be any pharmaceutically acceptable chelating agent. Chelating agents include ethylenediaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid and derivatives thereof. Citric acid also is known as citric acid monohydrate. Derivatives of citric acid include anhydrous citric acid and trisodiumcitrate-dihydrate. Still other chelating agents include niacinamide and derivatives thereof and sodium desoxycholate and derivatives thereof.

In some embodiments, a compound comprising a fibrin-binding peptide linked to an anticoagulant, e.g., a heparin-CREKA compound, is formulated with an antioxidant. The antioxidant may be any pharmaceutically acceptable antioxidant. Antioxidants are well known to those of ordinary skill in the art and include materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, and sodium sulfite. Such materials are typically added in ranges from 0.01 to 2.0%.

In some embodiments, a compound comprising a fibrin-binding peptide linked to an anticoagulant, e.g., a heparin-CREKA compound, is formulated with a cryoprotectant. The cryoprotecting agent may be any pharmaceutically acceptable cryoprotecting agent. Common cryoprotecting agents include histidine, polyethylene glycol, polyvinyl pyrrolidine, lactose, sucrose, mannitol, and polyols.

In some embodiments, a compound comprising a fibrin-binding peptide linked to an anticoagulant, e.g., a heparin-CREKA compound, is formulated with an isotonicity agent. The isotonicity agent can be any pharmaceutically acceptable isotonicity agent. This term is used in the art interchangeably with iso-osmotic agent, and is known as a compound which is added to the pharmaceutical preparation to increase the osmotic pressure, e.g., in some embodiments to that of 0.9% sodium chloride solution, which is iso-osmotic with human extracellular fluids, such as plasma. Preferred isotonicity agents are sodium chloride, mannitol, sorbitol, lactose, dextrose and glycerol.

The pharmaceutical preparation may optionally comprise a preservative. Common preservatives include those selected from the group consisting of chlorobutanol, parabens, thimerosol, benzyl alcohol, and phenol. Suitable preservatives include but are not limited to: chlorobutanol (0.30.9% W/V), parabens (0.01-5.0%), thimerosal (0.004-0.2%), benzyl alcohol (0.5-5%), phenol (0.1-1.0%), and the like.

In some embodiments, a compound comprising a fibrin-binding peptide linked to an anticoagulant, e.g., a heparin-CREKA compound, is formulated with a humectant to provide a pleasant mouth-feel in oral applications. Humectants known in the art include cholesterol, fatty acids, glycerin, lauric acid, magnesium stearate, pentaerythritol, and propylene glycol.

In some embodiments, an emulsifying agent is included in the formulations, for example, to ensure complete dissolution of all excipients, especially hydrophobic components such as benzyl alcohol. Many emulsifiers are known in the art, e.g., polysorbate 60.

For some embodiments related to oral administration, it may be desirable to add a pharmaceutically acceptable flavoring agent and/or sweetener. Compounds such as saccharin, glycerin, simple syrup, and sorbitol are useful as sweeteners.

In some embodiments, a compound comprising a fibrin-binding peptide linked to an anticoagulant, e.g., a heparin-CREKA compound, is administered in a pharmaceutically effective amount. In some embodiments, a compound comprising a fibrin-binding peptide linked to an anticoagulant, e.g., a heparin-CREKA compound, is administered in a therapeutically effective dose.

The dosage amount and frequency are selected to create an effective level of the compound without substantially harmful effects. When administered orally or intravenously, the dosage will generally range from 0.001 to 10,000 mg/kg/day or dose (e.g., 0.01 to 1000 mg/kg/day or dose; 0.1 to 100 mg/kg/day or dose).

For example, the usual adult dose for cardiovascular maladies such as, e.g., deep vein thrombosis, pulmonary embolism, myocardial infarction, angina pectoris, and some anticoagulation therapies is approximately one or more of 1000 to 10,000 units, 100 to 5000 units/per hour, and/or 10-100 units/kg subject mass. As another example, a typical adult dose of heparin for thrombotic/thromboembolic disorders approximately is 100 units/mL every 6 to 8 hours for catheters and peripheral heparin locks. In some embodiments of the present technology relating to a fibrin-binding peptide linked to an anticoagulant, e.g., a heparin-CREKA compound, the dosage of the fibrin-binding peptide linked to an anticoagulant, e.g., a heparin-CREKA compound, is lower (either for each dose administered or for the total dose administered over a particular time period) than the dosage of heparin that would be used for the same indication.

Methods of administering a pharmaceutically effective amount include, without limitation, administration in parenteral, oral, intraperitoneal, intranasal, topical, sublingual, rectal, and vaginal forms. Parenteral routes of administration include, for example, subcutaneous, intravenous, intramuscular, intrastemal injection, and infusion routes.

In some embodiments, a single dose of a fibrin-binding peptide linked to an anticoagulant, e.g., a heparin-CREKA compound, is administered to a subject. In other embodiments, multiple doses are administered over two or more time points, separated by hours, days, weeks, etc. In some embodiments, compounds are administered over a long period of time (e.g., chronically), for example, for a period of months or years (e.g., 1, 2, 3, 4, 5, 6, 7, 8, 9, 10, 11, 12, or more months or years; for the subject's lifetime). In such embodiments, compounds may be taken on a regular scheduled basis (e.g., daily, weekly, etc.) for the duration of the extended period.

For intravenous administration, pharmaceutical compositions may be given as a bolus, as two or more doses separated in time, or as a constant or non-linear flow infusion. Pharmaceutical dosage forms suitable for intravenous or subcutaneous administration of such pharmaceutical compositions can involve a sterile solution or a lyophilized powder. In the case of a lyophilized powder, the dosage forms can be reconstituted with water or saline for injection before administration to a patient.

The technology also relates to methods of treating a subject with a fibrin-binding peptide linked to an anticoagulant, e.g., a heparin-CREKA compound. According to another aspect of the technology, a method is provided for treating a subject in need of such treatment with an effective amount of a fibrin-binding peptide linked to an anticoagulant, e.g., a heparin-CREKA compound. The method involves administering to the subject an effective amount of a fibrin-binding peptide linked to an anticoagulant, e.g., a heparin-CREKA compound, in any one of the pharmaceutical preparations described above, detailed herein, and/or set forth in the claims. The subject can be any subject in need of such treatment. In the foregoing description, the technology is in connection with a fibrin-binding peptide linked to an anticoagulant, e.g., a heparin-CREKA compound, or a salt thereof. Such salts include, but are not limited to, bromide salts, chloride salts, iodide salts, carbonate salts, and sulfate salts. It should be understood, however, that the a fibrin-binding peptide linked to an anticoagulant, e.g., a heparin-CREKA compound, is a member of a class of compounds and the technology is intended to embrace pharmaceutical preparations, methods, and kits containing related derivatives within this class.

Some embodiments are related to combination therapies, e.g., methods comprising treating a subject with a fibrin-binding peptide linked to an anticoagulant, e.g., a heparin-CREKA compound, and one or more other drugs and/or bioactive agents, e.g., another anticoagulant, antiplatelet, thrombolytic, or fibrinolytic drug.

In some embodiments, combination therapies comprise use of an antiplatelet drug that inhibits platelet function, adhesion, activation, or aggregation. In addition, antiplatelet agents may inhibit secretion of prothrombotic or platelet activation factors and inhibit the recruitment and activation of monocytes. Examples include a GPIIb/IIIa receptor inhibitor, ADP receptor (e.g., P2Y₁ and P2Y₁₂) inhibitor, thrombin receptor (e.g., PAR-1 and PAR-4) inhibitor, CD40 inhibitor, CD40 L (CD40 ligand) inhibitor, Gas6 inhibitor, Gas6 receptor axl inhibitor, Gas 6 receptor inhibitor Sky, Gas6 receptor Mer inhibitor, P-selectin inhibitor, P-selectin receptor PSGL-1 inhibitor, thromboxane inhibitor, synthetase inhibitor, fibrinogen receptor antagonist, prostacyclin mimetic, phosphodiesterase inhibitor, RANTES inhibitor, phosphoinositide-3-kinase (PI(3)K) isoform β inhibitor, phosphoinositide-3-kinase (PI(3)K) isoform y inhibitor, eptifibatide, tirofiban, ticlopidine, and clopidogrel.

In some embodiments, combination therapies comprise use of a thrombolytic to affect, directly or indirectly, an enzyme, polypeptide, or cofactor in the plasmin-mediated fibrinolysis cascade, such as by activating certain enzymes (e.g., lytic enzymes or activators of lytic enzymes) or by inhibiting the inhibitors of certain enzymes (e.g., inhibiting lytic enzyme inhibitors). In some embodiments, thrombolytics for use in the present invention include tissue plasminogen activator (natural or recombinant), urokinase, plasminogen activators (e.g., vampire bat plasminogen activator), streptokinase, staphylokinase, prourokinase, anisolated streptokinase plasminogen activator complex (ASPAC), and animal salivary gland plasminogen activator.

In yet further embodiments, combination therapies comprise use of one or more drugs or agents that are, e.g., an antibiotic, immunomodulatory drug, steroid, analgesic, chemotherapeutic, antitumor agent, cytotoxic, radiopharmaceutical,

In some embodiments, a subject is tested to assess the presence, the absence, or the level of clotting and/or coagulation. Such testing is performed, e.g., by assaying or measuring a biomarker, a metabolite, a physical symptom, an indication, etc., to determine the risk of or the presence of clotting and/or coagulation. In some embodiments, a quantitative score is determined. In some embodiments, the subject is treated with a fibrin-binding peptide linked to an anticoagulant, e.g., a heparin-CREKA compound, based on the outcome of the test. Accordingly, in some embodiments, a subject is tested for clotting and/or coagulation and then treated for clotting and/or coagulation based on the test results. In some embodiments, a subject is treated for clotting and/or coagulation and then tested for clotting and/or coagulation to assess the efficacy of the treatment. In some embodiments, a subsequent clotting and/or coagulation treatment is adjusted based on a test result, e.g., the dosage amount, dosage schedule, identity of the drug, etc. is changed. In some embodiments, a patient is tested, treated, and then tested again to monitor the response to therapy and/or change the therapy. In some embodiments, cycles of testing and treatment may occur without limitation to the pattern of testing and treating (e.g., test/treat, treat/test, test/treat/test, treat/test/treat, test/treat/test/treat, test/treat/test/treat/test, test/treat/test/test/treat/treat/treat/test, test/treat/treat/test/treat/treat, etc), the periodicity, or the duration of the interval between each testing and treatment phase.

Some embodiments relate to the production of a compound comprising a fibrin-binding peptide linked to an anticoagulant. For example, in some embodiments a compound comprising a fibrin-binding peptide linked to an anticoagulant is produced by a two-step process. In some embodiments, the first step comprises conjugating a bifunctional linker (e.g., N-β-maleimidopropionic acid hydrazide, BMPH) to the anticoagulant (e.g., using 1-ethyl-3-(-3-dimethylaminopropyl) carbodiimide hydrochloride (EDC) in a carbodiimide reaction). Next, the second step comprises linking the fibrin-binding peptide (e.g., Cys-Arg-Glu-Lys-Ala, CREKA) to the linker, e.g., using a thiol-maleimide click reaction.

Embodiments of the technology comprise embodiments of reaction mixtures, e.g., reaction mixtures comprising a bifunctional linker (e.g., N-β-maleimidopropionic acid hydrazide, BMPH) and an anticoagulant. Some embodiments provide reaction mixtures comprising a bifunctional linker, an anticoagulant, and a carbodiimide (e.g., using 1-ethyl-3-(-3-dimethylaminopropyl) carbodiimide hydrochloride (EDC)). Some embodiments of reaction mixtures comprise a bifunctional linker linked to an anticoagulant. Some embodiments of reaction mixtures comprise a bifunctional linker linked to an anticoagulant and further comprise an unlinked fibrin-binding peptide (e.g., a CREKA peptide). Some embodiments of reaction mixtures comprise a bifunctional linker linked to an anticoagulant, an unlinked fibrin-binding peptide (e.g., a CREKA peptide), and a bifunctional linker linked to an anticoagulant and linked to a fibrin-binding peptide. Related embodiments relate to reaction intermediates such as a composition comprising, e.g., a bifunctional linker linked to an anticoagulant as described herein.

Although the disclosure herein refers to certain illustrated embodiments, it is to be understood that these embodiments are presented by way of example and not by way of limitation.

### EXAMPLES

Example 1—Synthesis of Heparin-CREKA

Heparin-CREKA is produced by a two-step process (Scheme 1). Briefly, the first step comprises conjugating a bifunctional linker (N-β-maleimidopropionic acid hydrazide, BMPH) to the heparin using 1-ethyl-3-(-3-dimethylaminopropyl) carbodiimide hydrochloride (EDC) in a carbodiimide reaction. Next, the second step comprises linking the fibrin binding peptide (Cys-Arg-Glu-Lys-Ala, CREKA) using a thiol-maleimide click reaction. All synthesis procedures are performed in aqueous solution with near-neutral pH conditions to preserve the heparin activity.

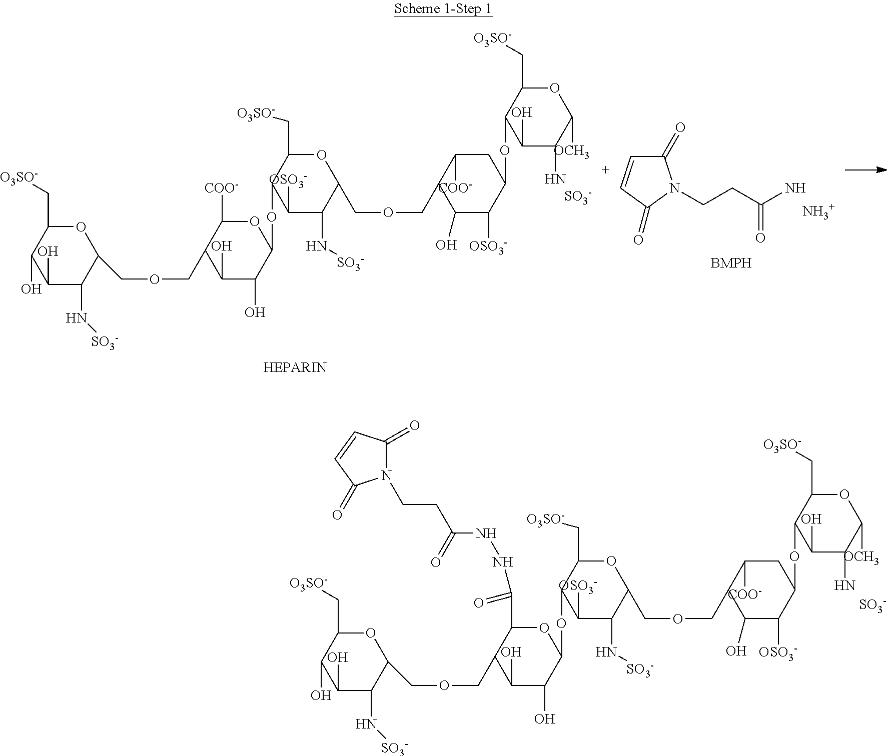

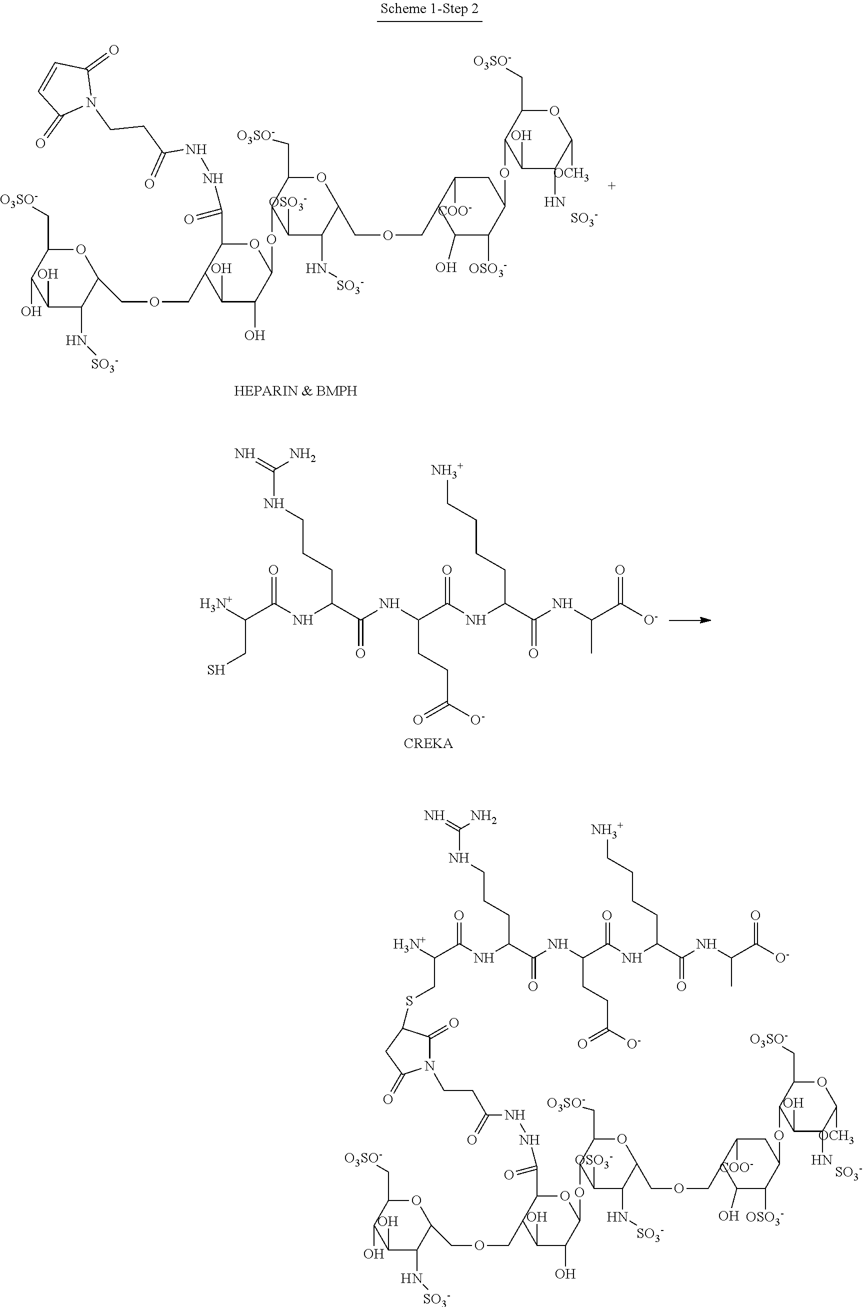

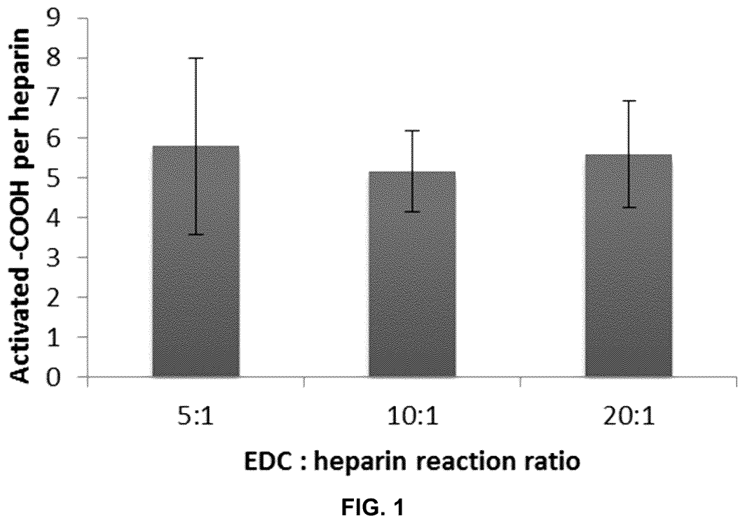

During the development of embodiments of the technology provided herein, experiments were conducted to maximize the efficiency of the first step of the synthetic scheme for producing heparin-CREKA. In particular, three ratios of EDC to heparin, 5 to 1, 10 to 1, and 20 to 1, were tested to determine the ratio that activates the most heparin carboxyl groups in Step 1 of the synthetic scheme. The efficacy of each ratio was determined using the 2,4,6-trinitrobenzene sulfonic acid (TNBS) assay. The TNBS assay quantifies the number of unreacted primary amines by producing a chromogenic product. The absorbance of the samples at 355 nm was measured using a plate reader. Based on the testing results, ratio 5:1 was chosen for the following studies (FIG. 1).

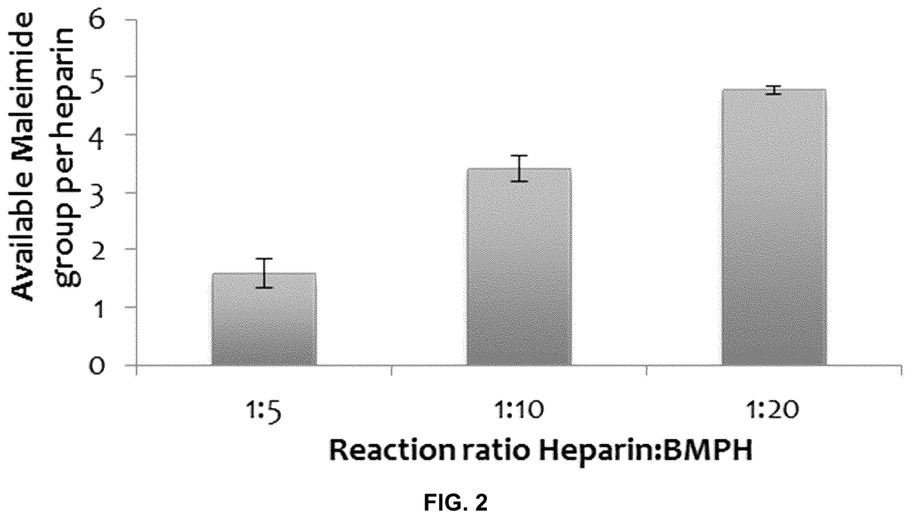

Next, experiments were conducted during the development of embodiments of the technology to maximize the efficiency of the second step of the synthetic scheme for producing heparin-CREKA. In particular, after the study performed above to maximize carboxyl group activation (e.g., using the identified ratio of EDC to heparin), the next step of adding the BMPH linker was studied. In particular, three ratios of BMPH to heparin, 5 to 1, 10 to 1, and 20 to 1, were tested. The efficacy of each ratio was determined using the Ellman's assay. The Ellman's assay quantifies the number of sulfhydryl groups by producing a chromogenic product, which was used to indicate the ratio that made available the most maleimide groups. A plate reader was used to determine the absorbance of the samples at 412 nm. The final reaction ratio was set to be 1 to 20 based on the test result (FIG. 2).

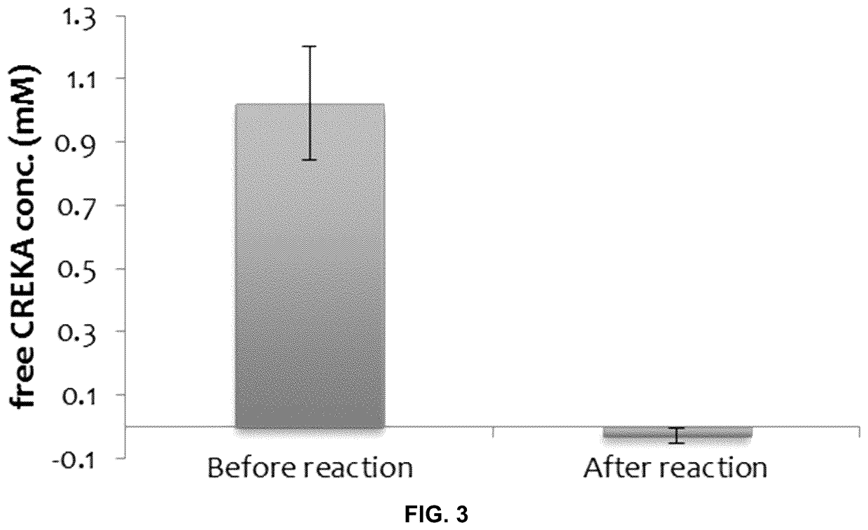

During the development of embodiments of the technology provided herein, the Ellman's assay was used again to determine the efficiency of the maleimide conjugation by measuring the concentration of CREKA remaining in the reaction mixture (FIG. 3). Heparin-CREKA was purified by dialyzing overnight with 3,800 MWCO dialysis membrane. The dialysis process was employed to remove reactants, byproducts and other components, including unreacted CREKA or excess reaction buffer, that were not conjugated to the heparin. The data indicated that little or no CREKA peptides remained unreacted, thus indicating that the efficiency of maleimide conjugation was very high, e.g., near 100%.

### Example 2—Heparin-CREKA Anti-Coagulant Activity

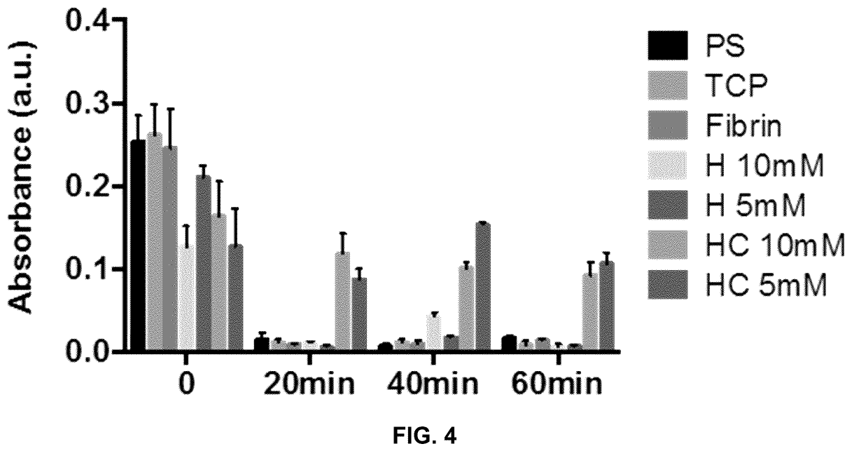

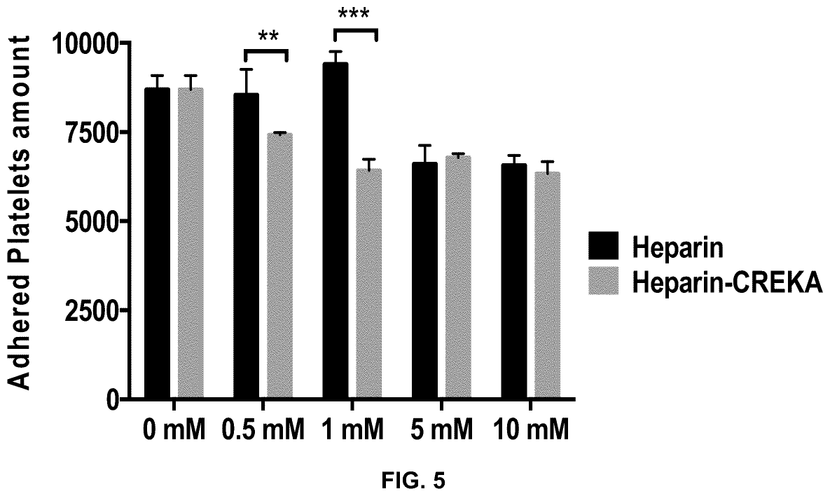

During the development of embodiments of the technology provided herein, experiments were conducted to test the anti-coagulant activity of embodiments of the heparin-CREKA compounds described herein. In particular, the heparin-CREKA compounds were evaluated by a whole blood clotting assay and a platelet adhesion assay (FIGS. 4 and 5). Pig whole blood was collected into acid citrate dextrose (ACD) anticoagulant. Fibrin coated 96-well plates were pre-treated with phosphate-buffered saline (PBS), heparin, or heparin-CREKA for 30 minutes at room temperature, then followed with three washes with PBS prior to the test. The wash removes unbound heparin or heparin-CREKA from the surfaces. Control experiments included tests of tissue culture treated and untreated polystyrene 96 well plates. The anti-coagulated whole blood samples were re-calcified with the addition of 10% (v/v) 0.1 m CaCl₂; then, 100 μl of re-calcified blood were then immediately incubated with the samples at room temperature. The coagulation process was monitored at each selected time point by adding 150 μl of deionized water into each well to lyse the unclotted red blood cells. The lysate was then transferred into a new plate and the absorbance was measured at a wavelength of 405 nm. Higher absorbance represents more unclotted red blood cells and better anti-coagulation effect. The anti-coagulant effect of heparin-CREKA was detected up to 60 minutes while the effect of the conventional heparin was undetectable 20 minutes after contacting the blood (FIG. 4).

Next, during the development of embodiments of the technology provided herein, experiments were conducted to test the anticoagulation activity of embodiments of the heparin-CREKA compounds described herein. In particular, a platelet adhesion assay was performed using platelet-rich plasma (PRP). Surfaces were treated with various concentrations of heparin or heparin-CREKA as described above. PRP, diluted 1:10 in PBS, was incubated at 37° C. for 60 minutes with the samples and gently rinsed with warm PBS. The number of adherent platelets was determined by detecting the amount of lactate dehydrogenase (LDH) present after cell lysis. Briefly, adherent platelets were lysed by incubation with 2% Triton-PSB buffer for 30 minutes at 37° C. A colorimetric substrate for LDH was added and incubated for 20 minutes at 37° C. The optical density was measured at a wavelength of 490 nm. A calibration curve was generated from a series of serial dilutions of a known platelet concentration and used to determine the number of adhered platelets. When compared to the bare fibrin surface (0 mM treated surfaces), heparin-CREKA treatment significantly reduced the amount of adhered platelets at a lower concentration than the addition of soluble heparin (FIG. 5).

### Example 3—Binding Specificity of Heparin-CREKA

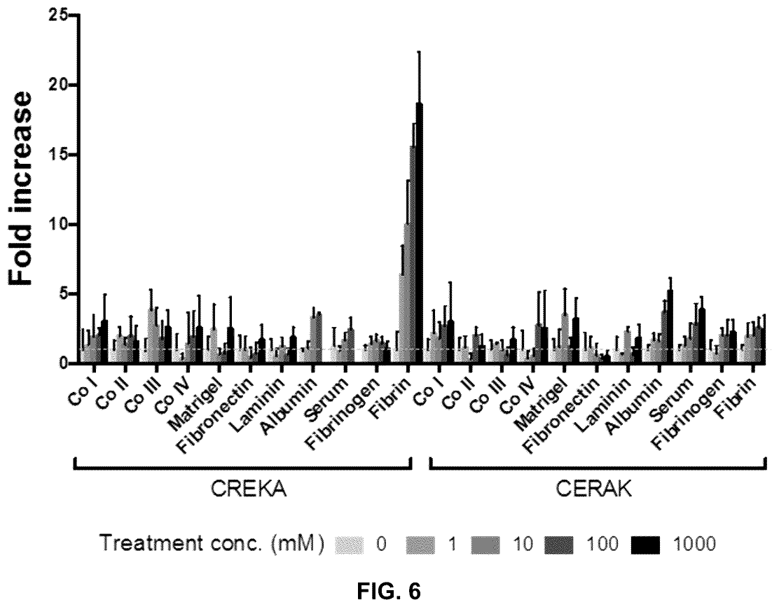

During the development of embodiments of the technology provided herein, experiments were conducted to assess the binding specificity of the CREKA peptides towards fibrin and non-target components. In these experiments, binding of biotinylated CREKA was compared with the binding of a scrambled inactive form of CREKA, e.g., CERAK. Proteins were applied to uncoated polystyrene surfaces at 10 μg/cm² and washed with PBS three times to remove unbound protein. A range of concentrations of biotinylated CREKA and biotinylated CERAK were incubated with the treated surfaces overnight, then washed three times the next day to remove the unbound peptides. The peptides remaining on the protein-coated surfaces were quantified using fluorophore-conjugated streptavidin. The data indicated that CREKA-fibrin binding is highly specific in a concentration-dependent relationship (FIG. 6). Additionally, data collected in the binding study indicated that CREKA binds very weakly with common blood plasma proteins such as albumin and fibrinogen. These properties of the heparin-CREKA compounds indicate that heparin-CREKA is appropriate for administration by intravenous injection.

### Example 4—in vivo Testing of Heparin-CREKA

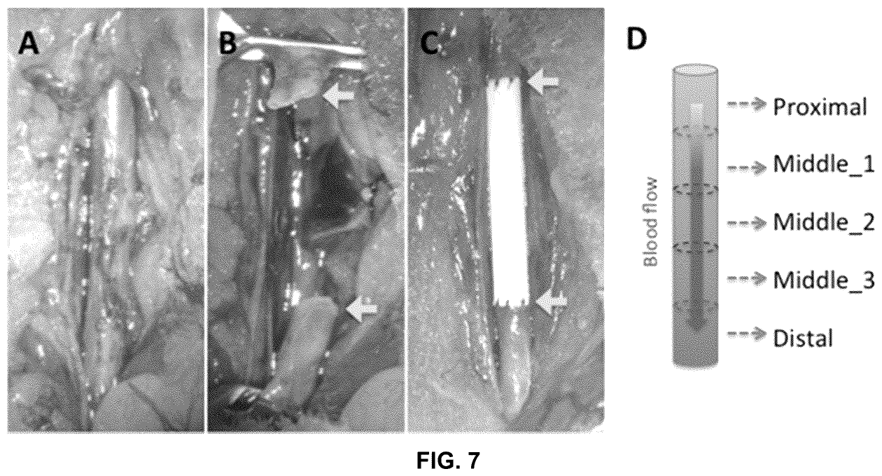

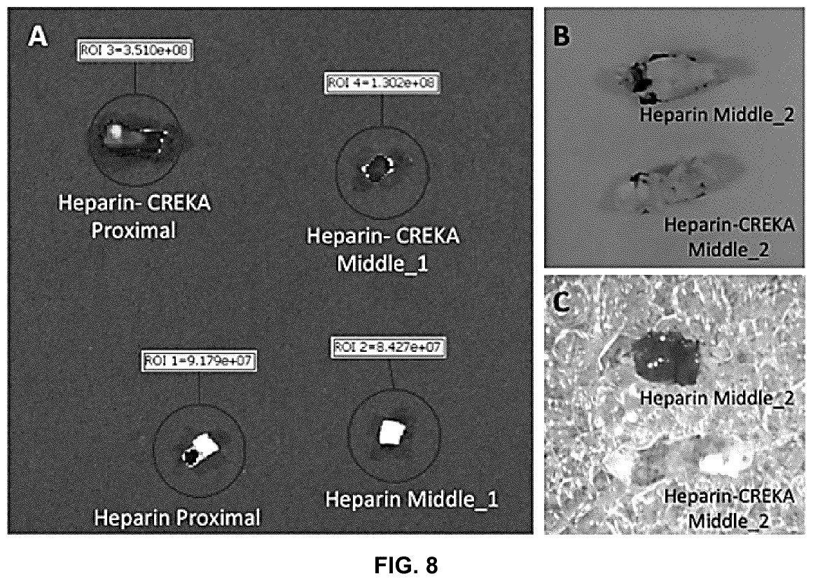

During the development of embodiments of the technology provided herein, experiments were conducted to evaluate the in vivo performance of the heparin-CREKA relative to conventional clinically used heparin. In particular, heparin-CREKA was studied in a rat abdominal aorta interposition model. Briefly, male Sprague Dawley rats (200-500 g) were anesthetized by a ketamine/xylazine cocktail. The abdomen of the anesthetized rat was entered through a midline incision. The abdominal aorta was exposed (FIG. 7, Panel A), clamped in both proximal and distal regions using micro-vessel clips, and then excised between the clamps (FIG. 7, Panel B). A segment (e.g., approximately 1 cm in length; 1.524±0.05 mm in diameter) of expanded polytetrafluoroethylene (ePTFE) was used as a graft. The graft was connected to the abdominal aorta using an end-to-end anastomosis technique with interrupted stitches at each end (FIG. 7, Panel C). Following reperfusion of blood flow through the graft, one dose of anti-coagulant (heparin or heparin-CREKA) was given systemically via intravenous injection. The incision was then closed in layers. 24 hours after the initial implantation surgeries and immediately prior to sacrifice of the test mice, a second dose of coagulant (heparin or heparin-CREKA) was administered systemically via intravenous injection. The grafts were then excised (including both anastomoses) and fixed in 4% formaldehyde solution. Each graft (1 cm long) was divided into five regions from the proximal to the distal end (proximal, middle_1, middle_2, middle_3, and distal, FIG. 8D), including the two anastomoses sites. Each segment was approximately 2 mm long.

The proximal and middle_1 pieces of the graft were first stained with avidin 594 nm, then imaged with an IVIS in vivo imaging system and embedded with optical coherence tomography (OCT) to detect and evaluate the biotinylated-CREKA peptides (FIG. 8, Panel A). The middle_2 pieces of each of the grafts were first stained with toluidine blue to detect heparin and then cut open and imaged under a dissection microscope (FIG. 8, Panel B). The middle_3 and distal segments were embedded in paraffin for histology (FIG. 8, Panel C). Data collected from these experiments indicated that: 1) the CREKA peptides were observed at the graft site in the heparin-CREKA treated group, thus indicating that the targeted binding activity of the heparin-CREKA targeted the heparin-CREKA to the grafted area when administrated systematically; 2) the toluidine blue staining of the grafts indicated toluidine staining of the heparin-CREKA grafts, which indicated the presence of additional carboxyl groups, presumably heparin, at the site; and 3) major clotting was observed in control grafts treated with conventional heparin while the grafts treated with heparin-CREKA remained clean, thus indicating the localized anti-coagulant effect of the heparin-CREKA compounds.

### Example 5—Dosing in Vivo

During the development of embodiments of the technology provided herein, experiments were conducted to study the in vivo binding and pharmacology response of the embodiments of heparin-CREKA. In particular, data were collected from three animal studies in which the dosing period was varied.

Study 1—animals were scarified 24 hours after the initial surgery and 2 doses of heparin-CREKA or control heparin-CERAK were administrated by intravenous injection during the 24 hour period. The first dose was administered immediately after the surgery (e.g., after re-establishing blood flow at the graft area). The second dose was administered 6 hours prior to euthanizing the animal. Imaging data were collected and analyzed to evaluate the anti-coagulant effect of heparin-CREKA at the graft site. The data indicated a significant improvement of the sites treated with heparin-CREKA relative to the sites treated with heparin-CERAK.

Study 2—animals were kept alive for 7 days after surgery and one dose of heparin-CREKA or control heparin-CERAK was administrated by intravenous injection immediately after the surgery (e.g., after re-establishing blood flow at the graft area). In contrast to Study 1, no additional dose was given after the first dose. Imaging data were collected and analyzed to evaluate the anti-coagulant effect of heparin-CREKA at the graft site. The data indicated only a very small difference in the effectiveness of the heparin-CREKA at the graft sites treated with heparin-CREKA relative to the graft sites treated with heparin-CERAK. Accordingly, these data suggest that a single dose treatment for 7 days may not be sufficient for some patients.

Study 3—animals were kept alive for 7 days after surgery and two doses of heparin-CREKA or control heparin-CERAK were administrated by intravenous injection during the 7 day period. The first dose was administered immediately after the surgery (e.g., after re-establishing blood flow at the graft area). The second dose was administered 6 hours before euthanizing the animal. Imaging data were collected and analyzed to evaluate the anti-coagulant effect of heparin-CREKA at the graft site. The data indicated a significant improvement of the graft sites treated with heparin-CREKA relative to the graft sites treated with heparin-CERAK. Accordingly, these data suggest that a second boost dose improves the effectiveness of the heparin-CERAK at graft sites in some patients, e.g., for a multi-day treatment regime.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A composition for reducing coagulation of blood, the composition comprising:
a) an anticoagulant; and
b) a fibrin-binding peptide covalently linked to the anticoagulant.

2. The composition of claim 1 wherein the anticoagulant is heparin.

3. The composition of claim 1 wherein the fibrin-binding peptide comprises the sequence CREKA (SEQ ID NO: 1).

4. The composition of claim 1 wherein the fibrin-binding peptide is a conservative variant of CREKA (SEQ ID NO: 1).

5. The composition of claim 1 wherein the fibrin-binding peptide is SREKA (SEQ ID NO: 2); CKEKA (SEQ ID NO: 3); CRDKA (SEQ ID NO: 4); CRERA (SEQ ID NO: 5);
CREKV (SEQ ID NO: 6); SKEKA (SEQ ID NO: 7); SRDKA (SEQ ID NO: 8); SRERA (SEQ ID NO: 9); SREKV (SEQ ID NO: 10); CKDKA (SEQ ID NO: 11); CKERA (SEQ ID NO: 12); CKEKV (SEQ ID NO: 13); CRDRA (SEQ ID NO: 14); CRDKV (SEQ ID NO: 15); or CRERV (SEQ ID NO: 16).

6. The composition of claim 1 wherein the fibrin-binding peptide is covalently linked to the anticoagulant by a linker.

7. The composition of claim 1 further comprising a physiologically appropriate solution for administration to a subject.

8. The composition of claim 1 further comprising a fibrin bound to the fibrin-binding peptide.

9. A system for reducing coagulation of blood, said system comprising a composition comprising:
a) an anticoagulant; and
b) a fibrin-binding peptide covalently linked to the anticoagulant.

10. The system of claim 9 further comprising a medical device.

11. The system of claim 10 wherein the medical device is in a patient and said composition reduces coagulation of blood at or near said medical device in the subject.

12. The system of claim 10 wherein the medical device is outside a patient and said composition reduces coagulation of blood contacting the medical device.

13. The system of claim 10 wherein the composition is administered to a subject and/or the medical device comprises the composition.

14. A method for treating a subject in need of anticoagulation therapy, the method comprising administering to the subject a composition comprising a fibrin-binding peptide covalently linked to an anticoagulant.

15. The method of claim 14 wherein the anticoagulant is heparin.

16. The method of claim 14 wherein the fibrin-binding peptide comprises the sequence CREKA (SEQ ID NO: 1).

17. The method of claim 14 wherein the composition comprises heparin-CREKA.

18. The method of claim 14 wherein the composition is administered at a dose that is lower than a dose of heparin appropriate for administration to the subject for the same therapy.

19. The method of claim 14 wherein treatment of the subject comprises use of a medical device that contacts blood of the subject.

20. The method of claim 19 wherein the medical device is implanted in the subject.

21. The method of claim 19 wherein the medical device is outside the subject.

22. The method of claim 19 further comprising testing the subject for coagulation.

23. A method for producing a fibrin-binding peptide covalently linked to an anticoagulant, the method comprising:
a) activating a carboxyl group on an anticoagulant;
b) covalently linking a linker to the activated carboxyl group; and
c) covalently linking a fibrin-binding peptide to the linker.

24. The method of claim 23 wherein the anticoagulant is heparin.

25. The method of claim 23 wherein the linker is a bifunctional linker comprising an amide and a maleimide.

26. The method of claim 23 wherein the fibrin-binding peptide is CREKA (SEQ ID NO: 1).

27. The method of claim 23 wherein activating the carboxyl group on the anticoagulant comprises use of a carbodiimide reaction.

28. The method of claim 23 wherein activating the carboxyl group on the anticoagulant comprises use of 1-ethyl-3-(-3-dimethylaminopropyl) carbodiimide hydrochloride.

29. The method of claim 23 wherein the bifunctional linker is N-β-maleimidopropionic acid hydrazide.

30. The method of claim 23 wherein covalently linking the fibrin-binding peptide to the linker comprises use of a thiol-maleimide click reaction.

31. The method of claim 23 wherein the fibrin-binding peptide is a conservative variant of CREKA (SEQ ID NO: 1).

32. The method of claim 33 wherein the fibrin-binding peptide is SREKA (SEQ ID NO: 2); CKEKA (SEQ ID NO: 3); CRDKA (SEQ ID NO: 4); CRERA (SEQ ID NO: 5); CREKV (SEQ ID NO: 6); SKEKA (SEQ ID NO: 7); SRDKA (SEQ ID NO: 8); SRERA (SEQ ID NO: 9); SREKV (SEQ ID NO: 10); CKDKA (SEQ ID NO: 11); CKERA (SEQ ID NO: 12); CKEKV (SEQ ID NO: 13); CRDRA (SEQ ID NO: 14); CRDKV (SEQ ID NO: 15); or CRERV (SEQ ID NO: 16).

33. A reaction mixture comprising an anticoagulant and a linker.

34. The reaction mixture of claim 33 wherein the anticoagulant comprises an activated group.

35. The reaction mixture of claim 33 wherein the anticoagulant comprises an activated carboxyl.

36. The reaction mixture of claim 33 wherein the linker is covalently attached to the anticoagulant.

37. The reaction mixture of claim 33 further comprising a fibrin-binding peptide.

38. The reaction mixture of claim 33 comprising an anticoagulant linked to a fibrin-binding peptide by a linker.

39. The reaction mixture of claim 33 wherein the anticoagulant is heparin.

40. The reaction mixture of claim 37 wherein the fibrin-binding peptide comprises the sequence CREKA (SEQ ID NO: 1).

41. The reaction mixture of claim 37 wherein the fibrin-binding peptide is a conservative variant of CREKA (SEQ ID NO: 1).

42. The reaction mixture of claim 37 wherein the fibrin-binding peptide is SREKA (SEQ ID NO: 2); CKEKA (SEQ ID NO: 3); CRDKA (SEQ ID NO: 4); CRERA (SEQ ID NO: 5); CREKV (SEQ ID NO: 6); SKEKA (SEQ ID NO: 7); SRDKA (SEQ ID NO: 8); SRERA (SEQ ID NO: 9); SREKV (SEQ ID NO: 10); CKDKA (SEQ ID NO: 11); CKERA (SEQ ID NO: 12); CKEKV (SEQ ID NO: 13); CRDRA (SEQ ID NO: 14); CRDKV (SEQ ID NO: 15); or CRERV (SEQ ID NO: 16).

43. The reaction mixture of claim 34 comprising 1-ethyl-3-(-3-dimethylaminopropyl) carbodiimide hydrochloride.

44. The reaction mixture of claim 33 wherein the linker is N-β-maleimidopropionic acid hydrazide.

45. The method of claim 14 wherein a plurality of doses of said composition comprising a fibrin-binding peptide covalently linked to an anticoagulant is administered to the subject.

46. The method of claim 45 wherein said plurality of doses is administered over a time period of two or more days.

47. The method of claim 45 wherein said plurality of doses comprises administering a first dose substantially immediately after surgery and a second dose is administered at least one day later.

48. A kit comprising a composition of claim 1.

49. A kit comprising a plurality of doses, wherein each dose comprises a composition of claim 1.

50. A kit comprising a plurality of doses, wherein a first dose comprises a composition of claim 1 and a second dose comprises a composition of claim 1 and said first and second doses comprise different amounts or concentrations of said composition.

51. A kit comprising a medical device, said medical device comprising a composition of claim 1.
